(19) United States
(12) Patent Application Publication
(10) Pub. No.: US 2020/0289095 A1
(43) Pub. Date: Sep. 17, 2020

(54) ULTRASOUND DIAGNOSTIC SYSTEM AND METHOD OF OPERATING ULTRASOUND DIAGNOSTIC SYSTEM

(71) Applicant: FUJIFILM Corporation, Tokyo (JP)

(72) Inventors: Hisashi ENDO, Kanagawa (JP); Masayuki TAKAHIRA, Kanagawa (JP)

(73) Assignee: FUJIFILM Corporation, Tokyo (JP)

(21) Appl. No.: 16/790,941

(22) Filed: Feb. 14, 2020

(30) Foreign Application Priority Data
Mar. 14, 2019 (JP) 2019-047124

(51) Int. Cl. A61B 8/08; A61B 8/00

(52) U.S. Cl. CPC A61B 8/5207; A61B 8/468; A61B 8/463; A61B 8/5246; A61B 8/4488; A61B 8/54

(57) ABSTRACT

There are provided an ultrasound diagnostic system and a method of operating the ultrasound diagnostic system of which operability for setting a measurement time is improved and which can automatically acquire an ultrasound image picked up at a point of time when a measurement time has elapsed.

In the ultrasound diagnostic system and the method of operating the ultrasound diagnostic system according to embodiments of the invention, a recording timing-management unit, which holds a plurality of kinds of recording patterns each of which includes a plurality of measurement times starting to be measured from a trigger timing, selects one recording pattern from the plurality of kinds of recording patterns according to an instruction given from a user; and an automatic storage control unit causes the image recording unit to record an ultrasound image, which is picked up at a point of time when each of the plurality of measurement times has elapsed, among a plurality of ultrasound images, which are continuously generated by an ultrasound image-generation unit, whenever each of the plurality of measurement times included in the one recording pattern elapses from the trigger timing.

### CROSS-REFERENCE TO RELATED APPLICATIONS

The present application claims priority under 35 U.S.C. § 119 to Japanese Patent Application No. 2019-047124, filed on Mar. 14, 2019. The above application is hereby expressly incorporated by reference, in its entirety, into the present application.

### BACKGROUND OF THE INVENTION

### 1. Field of the Invention

The present invention relates to an ultrasound diagnostic system that allows the state of a portion to be observed present in the body of an examinee to be observed using ultrasound, and a method of operating the ultrasound diagnostic system.

### 2. Description of the Related Art

For example, an ultrasound endoscope system has a main purpose to observe the pancreas, the gallbladder, or the like through the alimentary canal; and causes an ultrasound endoscope, which includes an endoscope observation part and an ultrasound observation part provided at the distal end thereof, to be inserted into the alimentary canal of an examinee and picks up the endoscopic images of the inside of the alimentary canal and the ultrasound images of a portion present outside the wall of the alimentary canal.

In the ultrasound endoscope system, an observation object-adjacent portion present in the alimentary canal is irradiated with illumination light emitted from an illumination unit provided at the distal end of the ultrasound endoscope, the reflected light of the illumination light is received by an image pickup unit provided at the distal end of the ultrasound endoscope, and endoscopic images are generated from the image pickup signals of the reflected light. Further, a plurality of ultrasound transducers provided at the distal end of the ultrasound endoscope are driven to transmit and receive ultrasound to and from a portion to be observed, such as an organ present outside the wall of the alimentary canal, and ultrasound images are generated from the reception signals of the ultrasound.

For the identification of whether the tumorous disease of the pancreas, the liver, and the like is, for example, a cancer or not or is benign or malignant, there is a case where a doctor gives a contrast medium to an examinee, observes the ultrasound images of a portion to be observed, and identifies whether disease is benign or malignant on the basis of the change of a brightness value over time.

In a case where such an observation is to be made, the change of a brightness value over time is analyzed on the basis of a time intensity curve (TIC) as disclosed in JP2001-178717A, JP2011-087629A, JP5905177B, and the like. TIC is a graph showing the change of the brightness value of a region of interest over time in a plurality of ultrasound images.

JP2001-178717A discloses an ultrasound diagnostic apparatus that measures time elapsed between the start of the giving of an ultrasound contrast medium and the end thereof with a stopwatch, causes the control of the measurement of the time to interlock with the control of the pickup or storage of ultrasound images, and simultaneously displays or stores the measurement time and the ultrasound images so that the measurement time and the ultrasound images are associated with each other. Further, JP2001-178717A discloses that TIC is created on the basis of the plurality of ultrasound images and is displayed on a monitor.

JP2011-087629A discloses an ultrasound image diagnostic apparatus that starts to measure time while interlocking with giving a contrast medium to an examinee and displays measurement time together with ultrasound image data. Further, JP2011-087629A discloses that a change in a brightness value of the ultrasound image data caused by giving a contrast medium is displayed.

JP5905177B discloses an ultrasound observation device that measures a brightness value or time as a measurement item related to the giving of a contrast medium and controls the change of setting of the quality of an ultrasound image on the basis of the results of the measurement. Further, JP5905177B discloses that a brightness change curve TIC (Time Intensity Curve) of the image data of a region to be observed from the start of the giving of a contrast medium is recorded and analyzed as data for TIC analysis.

### SUMMARY OF THE INVENTION

For example, a plurality of ultrasound images continuously generated are acquired as a video and the brightness value of the region of interest is calculated from each of the plurality of ultrasound images acquired as a video, so that the graph of TIC is created. In a case where a certain amount of time has elapsed after a contrast medium is given, brightness suddenly rises at a point of time when the contrast medium reaches a region of interest. After that, brightness gradually falls. The curve of a graph in a case where brightness falls is changed according to the characteristics of disease and the like, and may suddenly fall or gently fall.

For example, after a contrast medium is given, various index values, such as the brightness value of a region of interest, a different in a brightness value between two measurement times, and a change rate of the brightness value, at any measurement time are calculated using TIC and are analyzed to make a diagnosis, such as whether or not disease is benign or malignant. However, work for acquiring a video for TIC and calculating and analyzing various index values is very complicated, which is to be burden to a doctor. Since there are also many cases where a diagnosis can be made for certain disease from contrast-enhanced ultrasound findings at two to about four measurement times, it is thought that a case where ultrasound images picked up at an appropriate point of time can be easily acquired is useful.

JP2001-178717A discloses that a plurality of ultrasound images are automatically acquired at regular intervals from the time when an initial ultrasound image is acquired by an operator's manual operation after a contrast medium is given to an examinee.

However, since there is a possibility that time when ultrasound images required to identify disease are obtained is changed depending on conditions, such as the gender, the age, the weight, the disease, and a portion to be observed of an examinee, it cannot be said that the plurality of automatically acquired ultrasound images are suitable to identify disease. Accordingly, a doctor needs to repeatedly set suitable measurement time that is determined from the above-mentioned conditions and the like, and tries to set various measurement times by trial and error in some cases.

However, since an operation for manually setting various measurement times to acquire a plurality of ultrasound images is very complicated and needs to be repeated a plurality of times by trial and error, there is a problem that this operation is very bothersome.

Accordingly, an object of the invention is to provide an ultrasound diagnostic system and a method of operating the ultrasound diagnostic system of which operability for setting a measurement time is improved and which can automatically acquire an ultrasound image picked up at a point of time when a measurement time has elapsed.

In order to achieve the object, an aspect of the invention provides an ultrasound diagnostic system comprising: an ultrasound image-generation unit that drives an ultrasound transducer to cause the ultrasound transducer to transmit and receive ultrasound and generates an ultrasound image from a reception signal of the ultrasound; an instruction acquisition device that acquires an instruction input from a user; a recording timing-management unit that holds a plurality of kinds of recording patterns each of which includes a plurality of measurement times starting to be measured from a trigger timing and selects one recording pattern from the plurality of kinds of recording patterns according to the instruction input from the user; an image recording unit that records at least one ultrasound image among a plurality of ultrasound images continuously generated by the ultrasound image-generation unit; and an automatic storage control unit that causes the image recording unit to record an ultrasound image, which is picked up at a point of time when each of the plurality of measurement times has elapsed, among the plurality of ultrasound images, which are continuously generated by the ultrasound image-generation unit, whenever each of the plurality of measurement times included in the one recording pattern elapses from the trigger timing.

Here, it is preferable that the automatic storage control unit receives a recording pattern generated on the basis of at least one of information about an examinee, information about a portion to be observed of the examinee, or information about setting of the ultrasound diagnostic system, which is input to a recording pattern-generation device disposed outside the ultrasound diagnostic system, from the recording pattern-generation device, and causes the image recording unit to record the ultrasound image using the recording pattern received from the recording pattern-generation device.

Further, it is preferable that the recording timing-management unit receives a recording pattern generated on the basis of at least one of information about an examinee, information about a portion to be observed of the examinee, or information about setting of the ultrasound diagnostic system, which is input to a recording pattern-generation device disposed outside the ultrasound diagnostic system, from the recording pattern-generation device, and holds the recording pattern received from the recording pattern-generation device.

It is preferable that the ultrasound diagnostic system further comprises a recording pattern-generation unit that generates a recording pattern on the basis of at least one of information about an examinee, information about a portion to be observed of the examinee, or information about setting of the ultrasound diagnostic system input according to the instruction input from the user, and the automatic storage control unit causes the image recording unit to record the ultrasound image using the recording pattern generated by the recording pattern-generation unit.

It is preferable that the ultrasound diagnostic system further comprises a recording pattern-generation unit that generates a recording pattern on the basis of at least one of information about an examinee, information about a portion to be observed of the examinee, or information about setting of the ultrasound diagnostic system input according to the instruction input from the user, and the recording timing-management unit holds the recording pattern generated by the recording pattern-generation unit.

It is preferable that the ultrasound diagnostic system further comprises a timer control unit that includes a timer and controls measurement of a time performed by the timer, and the automatic storage control unit uses, as the trigger timing, a timing to start the measurement of a time performed by the timer.

It is preferable that the ultrasound diagnostic system further comprises an image playback unit that causes a monitor to simultaneously display a plurality of the ultrasound images, which are recorded in the image recording unit, side by side.

Further, it is preferable that the image playback unit causes the monitor to display a thumbnail image of the ultrasound image recorded in the image recording unit whenever the ultrasound image is recorded in the image recording unit.

Furthermore, it is preferable that the image playback unit causes the monitor to display a graph showing a relationship between a time elapsed from the trigger timing and an average brightness value of a region of interest of the ultrasound image.

Moreover, it is preferable that at least one recording pattern of the plurality of kinds of recording patterns held by the recording timing-management unit includes a determination flag that causes the ultrasound diagnostic system to determine a recording timing to record the ultrasound image in the image recording unit.

It is preferable that the ultrasound diagnostic system further comprises an image analysis unit that includes a temporary storage area, stores the ultrasound images in the temporary storage area from the trigger timing, analyzes the ultrasound images stored in the temporary storage area, and determines the recording timing on the basis of results of the analysis, and the automatic storage control unit causes the image recording unit to record an ultrasound image, which is picked up at the recording timing determined on the basis of the results of the analysis, among the ultrasound images stored in the temporary storage area in a case where the determination flag is included in the one recording pattern.

Further, it is preferable that the image analysis unit determines at least one of a recording timing when an average brightness value of a region of interest of the ultrasound image is maximum, a recording timing when the average brightness value is minimum, a recording timing when an amount of change in the average brightness value between two ultrasound images temporally continuous is maximum, a recording timing when a variance value of a brightness value of the region of interest is maximum, a recording timing when the variance value of the brightness value is minimum, or a recording timing when an amount of change in the variance value of the brightness value between two ultrasound images temporally continuous is maximum.

Furthermore, it is preferable that the automatic storage control unit sets an initial value of the region of interest according to a type of a probe used in the ultrasound diagnostic system.

It is preferable that the recording timing-management unit further holds a new recording pattern created according to the instruction input from the user.

It is preferable that the recording timing-management unit further changes at least one of the plurality of measurement times included in the one recording pattern according to the instruction input from the user.

Further, another aspect of the invention provides a method of operating an ultrasound diagnostic system comprising: a step of causing an ultrasound image-generation unit to drive an ultrasound transducer to cause the ultrasound transducer to transmit and receive ultrasound and causing the ultrasound image-generation unit to generate an ultrasound image from a reception signal of the ultrasound; a step of causing a recording timing-management unit, which holds a plurality of kinds of recording patterns each of which includes a plurality of measurement times starting to be measured from a trigger timing, to select one recording pattern from the plurality of kinds of recording patterns according to an instruction input from a user; and a step of causing an automatic storage control unit to cause an image recording unit to record an ultrasound image, which is picked up at a point of time when each of the plurality of measurement times has elapsed, among a plurality of ultrasound images, which are continuously generated by the ultrasound image-generation unit, whenever each of the plurality of measurement times included in the one recording pattern elapses from the trigger timing.

Here, it is preferable that at least one of information about an examinee, information about a portion to be observed of the examinee, or information about setting of the ultrasound diagnostic system is input to a recording pattern-generation device disposed in the ultrasound diagnostic system, a recording pattern generated on the basis of at least the one of the information about the examinee, the information about the portion to be observed of the examinee, or the information about the setting of the ultrasound diagnostic system is received from the recording pattern-generation device, and the image recording unit is caused to record the ultrasound image using the recording pattern received from the recording pattern-generation device.

Further, it is preferable that a timing to start measurement of a time performed by the timer, which is controlled by a timer control unit including the timer, is used as the trigger timing.

It is preferable that the method of operating an ultrasound diagnostic system further comprises a step of causing an image playback unit to cause a monitor to simultaneously display a plurality of ultrasound images, which are recorded in the image recording unit, side by side.

Further, it is preferable that the monitor is caused to display a thumbnail image of the ultrasound image recorded in the image recording unit whenever the ultrasound image is recorded in the image recording unit.

Furthermore, it is preferable that the monitor is caused to display a graph showing a relationship between a time elapsed from the trigger timing and an average brightness value of a region of interest of the ultrasound image.

Further, it is preferable that at least one recording pattern of the plurality of kinds of recording patterns held by the recording timing-management unit includes a determination flag that causes the ultrasound diagnostic system to determine a recording timing to record the ultrasound image in the image recording unit.

It is preferable that the method of operating an ultrasound diagnostic system comprises a step of causing an image analysis unit, which includes a temporary storage area, to store the ultrasound images in the temporary storage area from the trigger timing, to analyze the ultrasound images stored in the temporary storage area, and to determine the recording timing on the basis of results of the analysis, and the image recording unit is caused to record an ultrasound image, which is picked up at the recording timing determined on the basis of the results of the analysis, among the ultrasound images stored in the temporary storage area in a case where the determination flag is included in the one recording pattern.

Further, it is preferable that at least one of a recording timing when an average brightness value of a region of interest of the ultrasound image is maximum, a recording timing when the average brightness value is minimum, a recording timing when the amount of change in the average brightness value between two ultrasound images temporally continuous is maximum, a recording timing when a variance value of a brightness value of the region of interest is maximum, a recording timing when the variance value of the brightness value is minimum, or a recording timing when the amount of change in the variance value of the brightness value between two ultrasound images temporally continuous is maximum is determined.

Furthermore, it is preferable that an initial value of the region of interest is set according to a type of a probe used in the ultrasound diagnostic system.

Moreover, it is preferable that a new recording pattern created according to the instruction input from the user is further held.

Moreover, it is preferable that at least one of a plurality of measurement times included in the one recording pattern is further changed according to the instruction input from the user.

Further, it is preferable that the instruction acquisition device, the timer control unit, the recording timing-management unit, the recording pattern-generation unit, the image analysis unit, the automatic storage control unit, and the image playback unit are hardware or processors executing programs.

A plurality of kinds of recording patterns including a plurality of elapsed times are held in the ultrasound diagnostic system according to the aspect of the invention according to gender, age, weight, disease, a portion to be observed, and the like. Since a user of the ultrasound diagnostic system can set a plurality of measurement times together by a simple operation for merely designating a desired recording pattern among the plurality of kinds of recording patterns, the user can automatically acquire an ultrasound image picked up at a point of time when each of the plurality of measurement times has elapsed from the trigger timing.

### BRIEF DESCRIPTION OF THE DRAWINGS

FIG. 1 is a diagram showing the schematic configuration of an ultrasound endoscope system according to an embodiment of the invention.

FIG. 2 is a plan view showing a distal end part of an insertion unit of an ultrasound endoscope and the periphery thereof.

FIG. 3 is a diagram showing the cross section of the distal end part of the insertion unit of the ultrasound endoscope taken along line I-I of FIG. 2.

FIG. 4 is a block diagram showing the configuration of an ultrasound observation device.

FIG. 5 is a flowchart showing the flow of diagnostic processing using the ultrasound endoscope system.

FIG. 6 is a flowchart showing the procedure of a diagnostic step of the diagnostic processing.

FIG. 7 is a conceptual diagram of an embodiment showing the screen of an operation panel of an operation console.

FIG. 8 is a flowchart of an embodiment showing the operation of an ultrasound diagnostic system in a case where ultrasound images are observed in a contrast mode.

FIG. 9 is a conceptual diagram of an embodiment showing an aspect where ultrasound images continuously generated are displayed on a monitor as a video in real time in a live mode.

FIG. 10 is a conceptual diagram of an embodiment showing an aspect where a plurality of ultrasound images recorded in an image recording unit are simultaneously displayed on the monitor side by side in a contrast mode.

FIG. 11 is a flowchart of an embodiment showing the operation of the ultrasound diagnostic system in a case where a recording timing to record an ultrasound image in the image recording unit is to be determined.

### DESCRIPTION OF THE PREFERRED EMBODIMENTS

An ultrasound endoscope system will be described in detail below as an embodiment (this embodiment) of an ultrasound diagnostic system of the invention with reference to preferred embodiments shown in accompanying drawings by way of example.

This embodiment is a representative embodiment of the invention, but is merely exemplary and does not limit the invention.

Further, a numerical range represented using “to” in this specification means that numerical values written in the front and rear of “to” are included as the lower limit and the upper limit.

Outline of Ultrasound Endoscope System

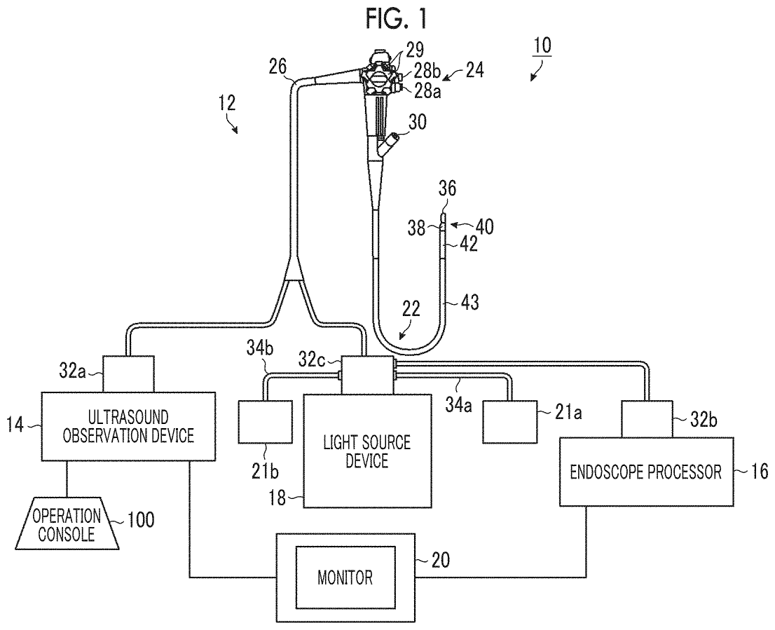

The outline of an ultrasound endoscope system 10 according to this embodiment will be described with reference to FIG. 1. FIG. 1 is a diagram showing the schematic configuration of the ultrasound endoscope system 10.

The ultrasound endoscope system 10 is used to make an observation (hereinafter, referred to as ultrasound diagnosis) of the state of a portion to be observed present in a patient's body, which is an examinee, using ultrasound. Here, the portion to be observed is a portion that is not easily examined from the surface side of the patient's body, and is, for example, the pancreas, the gallbladder, or the like. In a case where the ultrasound endoscope system 10 is used, the state of a portion to be observed and whether or not abnormality occurs at a portion to be observed can be diagnosed with ultrasound through the alimentary canal, such as the gullet, the stomach, the duodenum, the small intestine, and the large intestine, which are the patient's body cavity.

The ultrasound endoscope system 10 is to acquire ultrasound images and endoscopic images. As shown in FIG. 1, the ultrasound endoscope system 10 includes an ultrasound endoscope 12, an ultrasound observation device 14, an endoscope processor 16, a light source device 18, a monitor 20, a water supply tank 21a, a suction pump 21b, and an operation console 100.

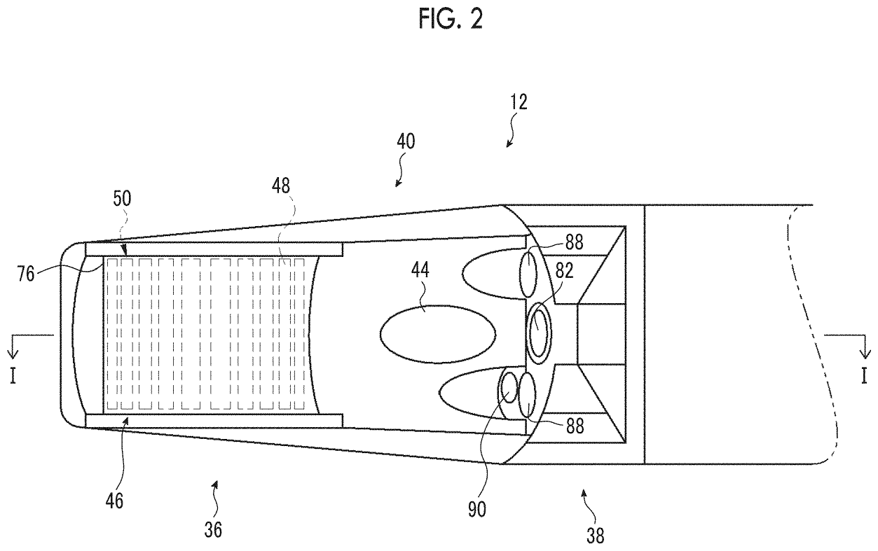

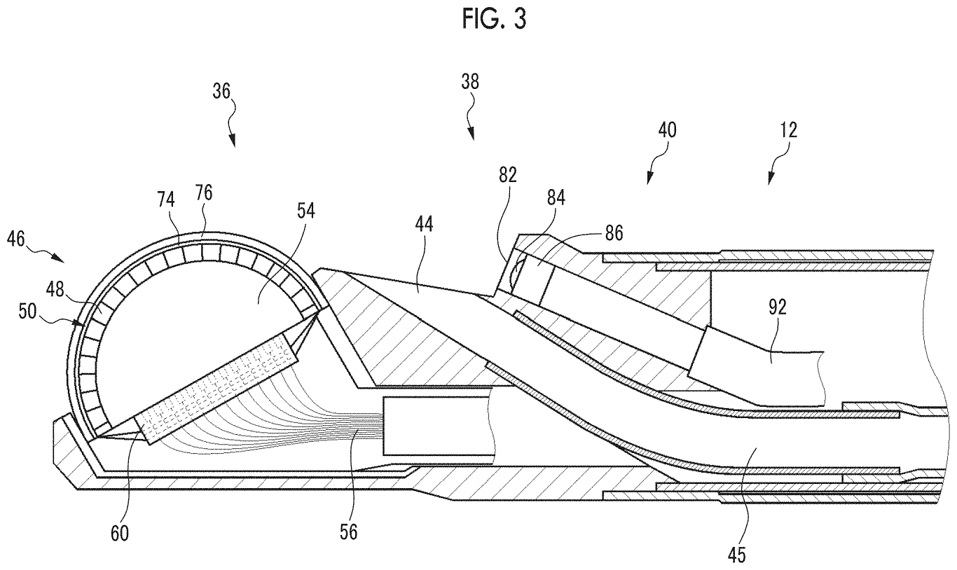

The ultrasound endoscope 12 comprises an insertion unit 22 that is to be inserted into the patient's body cavity, an operation unit 24 that is to be operated by an operator (the user of the ultrasound endoscope system 10), such as a doctor or a technician, and an ultrasound transducer unit 46 (see FIGS. 2 and 3) that is mounted on a distal end part 40 of the insertion unit 22. An operator acquires endoscopic images and ultrasound images by the function of the ultrasound endoscope 12.

Here, “endoscopic images” are images that are obtained in a case where the images of the inner wall of the patient's body cavity are picked by an optical method. Further, “ultrasound images” are images that are obtained in a case where reflected waves (echoes) of ultrasound transmitted toward a portion to be observed from the inside of the patient's body cavity are received and the reception signals are converted into images.

The ultrasound endoscope 12 will be described in detail later.

The ultrasound observation device 14 is connected to the ultrasound endoscope 12 through a universal cord 26 and a connector 32a for ultrasound provided at the end portion of the ultrasound observation device 14. The ultrasound observation device 14 controls the ultrasound transducer unit 46 of the ultrasound endoscope 12 to cause the ultrasound transducer unit 46 to transmit ultrasound. Further, the ultrasound observation device 14 converts reception signals, which are obtained in a case where the ultrasound transducer unit 46 receives the reflected waves (echoes) of the transmitted ultrasound, into images to generate ultrasound images.

The ultrasound observation device 14 will be described in detail later.

The endoscope processor 16 is connected to the ultrasound endoscope 12 through the universal cord 26 and a connector 32b for an endoscope provided at the end portion of the endoscope processor 16. The endoscope processor 16 acquires the image data of an observation object-adjacent portion of which the images are picked up by the ultrasound endoscope 12 (in detail, a solid image pickup element 86 to be described later), and performs predetermined image processing on the acquired image data to generate endoscopic images.

Here, “observation object-adjacent portion” is a portion of the inner wall of the patient's body cavity that is present at a position adjacent to a portion to be observed.

In this embodiment, the ultrasound observation device 14 and the endoscope processor 16 are formed of two devices (computers) that are provided separately from each other. However, the ultrasound observation device 14 and the endoscope processor 16 are not limited thereto, and both the ultrasound observation device 14 and the endoscope processor 16 may be formed of one device.

The light source device 18 is connected to the ultrasound endoscope 12 through the universal cord 26 and a connector 32c for a light source provided at the end portion of the light source device 18. In a case where the images of an observation object-adjacent portion are to be picked up by the ultrasound endoscope 12, the light source device 18 applies specific wavelength light or white light formed of three primary color lights, that is, red light, green light, and blue light. Light applied by the light source device 18 propagates through light guides (not shown), which are provided in the universal cord 26, and the ultrasound endoscope 12 and is emitted from the ultrasound endoscope 12 (in detail, illumination windows 88 to be described later). Accordingly, the observation object-adjacent portion is illuminated with the light applied from the light source device 18.

The monitor 20 is connected to the ultrasound observation device 14 and the endoscope processor 16, and displays the ultrasound images generated by the ultrasound observation device 14 and the endoscopic images generated by the endoscope processor 16. A method of switching and displaying any one of the ultrasound images or the endoscopic images on the monitor 20 or a method of simultaneously displaying both the ultrasound images and the endoscopic images may be used as a method of displaying the ultrasound images and the endoscopic images.

The ultrasound images and the endoscopic images are displayed on one monitor 20 in this embodiment, but a monitor for displaying the ultrasound images and a monitor for displaying the endoscopic images may be provided separately. Further, the ultrasound images and the endoscopic images may be displayed in a display aspect other than the monitor 20, for example, in an aspect where the images are displayed on the display of a terminal that is being carried by an operator.

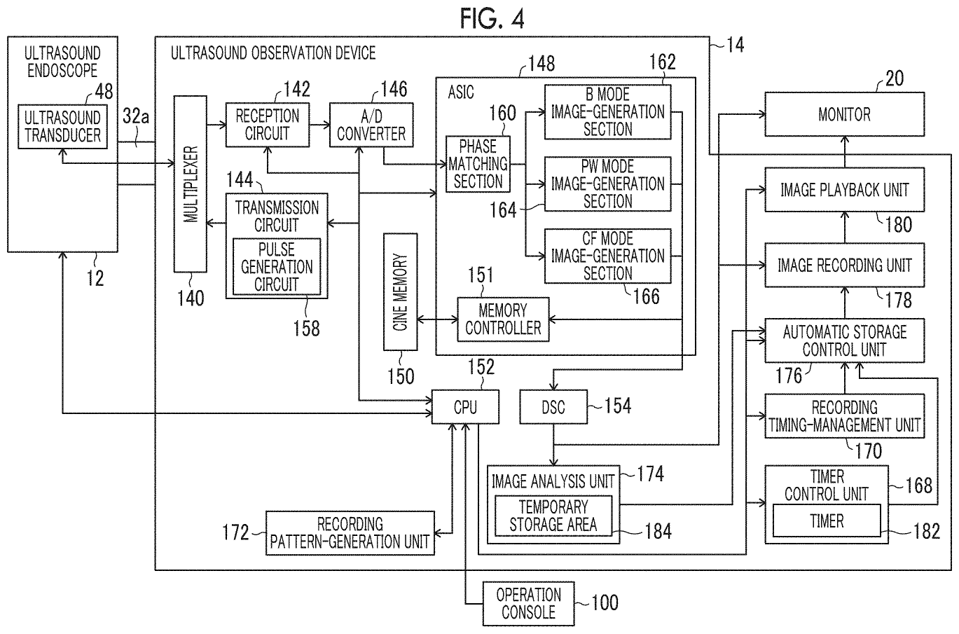

The operation console 100 is an example of an instruction acquisition device that acquires an instruction input from an operator (user), and is a device that is provided to allow an operator to input necessary information at the time of ultrasound diagnosis, to instruct the ultrasound observation device 14 to start ultrasound diagnosis, or the like. The operation console 100 includes, for example, a keyboard, a mouse, a trackball, a touch pad, a touch panel, and the like. In a case where the operation console 100 is operated, a CPU (control circuit) 152 (see FIG. 4) of the ultrasound observation device 14 controls the respective units (for example, a reception circuit 142 and a transmission circuit 144 to be described later) of the device according to the contents of the operation of the operation console 100.

Specifically, an operator inputs examination information (for example, examination order information including a date, an order number, and the like, and patient information including a patient ID, a patient name, and the like) through the operation console 100 in a stage where ultrasound diagnosis is not yet started. In a case where an operator instructs the ultrasound observation device 14 to start ultrasound diagnosis through the operation console 100 after the input of the examination information is completed, the CPU 152 of the ultrasound observation device 14 controls the respective units of the ultrasound observation device 14 on the basis of the input examination information so that ultrasound diagnosis is performed.

Further, an operator can set various control parameters through the operation console 100 at the time of performing ultrasound diagnosis. Examples of the control parameters include the result of selection of a live mode and a freeze mode, the set value of a display depth (depth), the result of selection of ultrasound image generation modes, and the like.

Here, “live mode” is a mode where ultrasound images (video) obtained at a predetermined frame rate are sequentially displayed (displayed in real time). “Freeze mode” is a mode where an image (static image) of one frame among ultrasound images (video) generated in the past is read out from a cine memory 150 to be described later and is displayed.

There are a plurality of ultrasound image generation modes that can be selected in this embodiment. Specifically, the plurality of ultrasound image generation modes include a brightness (B) mode, a color flow (CF) mode, a pulse wave (PW) mode, a contrast mode, and the like. The B mode is a mode where the amplitude of an ultrasound echo is converted into brightness and a tomographic image is displayed. The CF mode is a mode where an average blood flow rate, a flow variation, the intensity or flow power of a flow signal, and the like are mapped to various colors and are superimposed and displayed on a B mode image. The PW mode is a mode where the speed of an ultrasound echo source (for example, a blood flow rate) detected on the basis of the transmission and reception of a pulse wave is displayed. The contrast mode is a mode where a contrast medium is given to a patient and a B mode image is displayed.

The above-mentioned ultrasound image generation modes are merely exemplary, and may further include modes other than the four kinds of modes having been described above, for example, an amplitude (A) mode, a motion (M) mode, and the like.

Configuration of Ultrasound Endoscope 12

Next, the configuration of the ultrasound endoscope 12 will be described with reference to FIGS. 1, 2, and 3 having been already mentioned. FIG. 2 is an enlarged plan view showing the distal end part of the insertion unit 22 of the ultrasound endoscope 12 and the periphery thereof. FIG. 3 is a cross-sectional view of the distal end part 40 of the insertion unit 22 of the ultrasound endoscope 12 taken along line I-I of FIG. 2.

The ultrasound endoscope 12 includes the insertion unit 22 and the operation unit 24 as described above. As shown in FIG. 1, the insertion unit 22 comprises a distal end part 40, a bendable part 42, and a soft part 43 that are arranged in this order from a distal end side (free end side). As shown in FIG. 2, the distal end part 40 is provided with an ultrasound observation part 36 and an endoscope observation part 38. As shown in FIG. 3, an ultrasound transducer unit 46 comprising a plurality of ultrasound transducers 48 is disposed at the ultrasound observation part 36.

Further, the distal end part 40 is provided with a treatment tool outlet 44 as shown in FIG. 2. The treatment tool outlet 44 is an outlet for a treatment tool (not shown), such as forceps, a puncture needle, or a diathermy knife. Furthermore, the treatment tool outlet 44 also functions as a suction port in a case where aspirates, such as blood and internal sordes, are to be sucked.

The bendable part 42 is a part that is connected to the proximal end side (a side opposite to a side where the ultrasound transducer unit 46 is provided) of the distal end part 40, and can be freely bent. The soft part 43 is a part that connects the bendable part 42 to the operation unit 24, has flexibility, and is provided to extend in an elongated shape.

A plurality of air/water supply pipe lines and a plurality of suction pipe lines are formed in each of the insertion unit 22 and the operation unit 24. In addition, a treatment tool channel 45 of which one end communicates with the treatment tool outlet 44 is formed in each of the insertion unit 22 and the operation unit 24.

Next, the ultrasound observation part 36, the endoscope observation part 38, the water supply tank 21a, the suction pump 21b, and the operation unit 24 among the components of the ultrasound endoscope 12 will be described in detail.

Ultrasound Observation Part 36

The ultrasound observation part 36 is a part that is provided to acquire ultrasound images, and is disposed on the distal end side in the distal end part 40 of the insertion unit 22. As shown in FIG. 3, the ultrasound observation part 36 comprises the ultrasound transducer unit 46, a plurality of coaxial cables 56, and a flexible printed circuit (FPC) 60.

The ultrasound transducer unit 46 corresponds to an ultrasound probe, transmits ultrasound in a patient's body cavity using an ultrasound transducer array 50 where a plurality of ultrasound transducers 48 to be described later are arranged, receives the reflected waves (echoes) of the ultrasound reflected by a portion to be observed, and outputs reception signals. The ultrasound transducer unit 46 according to this embodiment is a convex ultrasound transducer unit, and transmits ultrasound radially (in the form of an arc). However, the kind (type) of the ultrasound transducer unit 46 is not particularly limited to the convex ultrasound transducer unit. As long as an ultrasound transducer unit can transmit and receive ultrasound, the ultrasound transducer unit may be another kind of ultrasound transducer unit and may be, for example, a radial ultrasound transducer unit, a linear ultrasound transducer unit, and the like.

The ultrasound transducer unit 46 has structure where a backing material layer 54, an ultrasound transducer array 50, an acoustic matching layer 74, and an acoustic lens 76 are laminated as shown in FIG. 3.

The ultrasound transducer array 50 is formed of a plurality of ultrasound transducers 48 that are arranged in the form of a one-dimensional array. In more detail, the ultrasound transducer array 50 has structure where N (for example, N=128) ultrasound transducers 48 are arranged in the shape of a convex curve at regular intervals in the axial direction of the distal end part 40 (the longitudinal direction of the insertion unit 22). The ultrasound transducer array 50 may have structure where a plurality of ultrasound transducers 48 are arranged in the form of a two-dimensional array.

Each of the N ultrasound transducers 48 has structure where electrodes are disposed on both surfaces of a piezoelectric element (piezoelectric body). Barium titanate (BaTiO₃), lead zirconate titanate (PZT), potassium niobate (KNbO₃), and the like are used for the piezoelectric element.

The electrodes are formed of individual electrodes (not shown) that are individually provided on the plurality of ultrasound transducers 48 and a transducer ground (not shown) that is common to the plurality of ultrasound transducers 48. Further, the electrodes are electrically connected to the ultrasound observation device 14 through the coaxial cables 56 and the FPC 60.

Pulsed drive voltages are supplied to the respective ultrasound transducers 48 from the ultrasound observation device 14 through the coaxial cable 56 as input signals (transmission signals). In a case where the drive voltages are applied to the electrodes of the ultrasound transducers 48, the piezoelectric elements extend and contract and the ultrasound transducers 48 are driven (vibrated). As a result, pulsed ultrasound is output from the ultrasound transducers 48. At this time, the amplitude of the ultrasound output from the ultrasound transducer 48 has a magnitude corresponding to intensity (output intensity) that is obtained in a case where the ultrasound transducer 48 outputs ultrasound. Here, output intensity is defined as the magnitude of the sound pressure of the ultrasound output from the ultrasound transducer 48.

Further, in a case where each ultrasound transducer 48 receives the reflected waves (echoes) of the ultrasound, each ultrasound transducer 48 is vibrated (driven) and the piezoelectric element of each ultrasound transducer 48 generates electrical signals. The electrical signals are output to the ultrasound observation device 14 from each ultrasound transducer 48 as the reception signals of the ultrasound. At this time, the magnitude (voltage value) of the electrical signal output from the ultrasound transducer 48 is a magnitude corresponding to receiving sensitivity that is obtained in a case where the ultrasound transducer 48 receives the ultrasound. Here, receiving sensitivity is defined as a ratio of the amplitude of the electrical signal, which is output from the ultrasound transducer 48 in a case where the ultrasound transducer 48 receives ultrasound, to the amplitude of the ultrasound transmitted from the ultrasound transducer 48.

In this embodiment, in a case where the N ultrasound transducers 48 are sequentially driven by an electronic switch, such as a multiplexer 140 (see FIG. 4), scanning using ultrasound is performed in a scanning range following the curved surface on which the ultrasound transducer array 50 is disposed, for example, a range within a distance of about several tens mm from the center of curvature of the curved surface. In more detail, in a case where B mode images (tomographic images) are to be acquired as the ultrasound images, drive voltages are supplied to m (for example, m=N/2) ultrasound transducers 48 (hereinafter, referred to as transducers to be driven), which continuously line up, among the N ultrasound transducers 48 as an open channel is selected by the multiplexer 140. Accordingly, the m transducers to be driven are driven and ultrasound is output from the respective transducers to be driven corresponding to the open channels. The ultrasound output from the m transducers to be driven is combined immediately, and the combined ultrasound (ultrasound beam) is transmitted to a portion to be observed. After that, each of the m transducers to be driven receives ultrasound (echoes) reflected by the portion to be observed, and outputs electrical signals (reception signals) corresponding to receiving sensitivity at that point of time.

Then, the series of processes (that is, the supply of drive voltages, the transmission and reception of ultrasound, and the output of electrical signals) are repeatedly performed while the positions of the transducers to be driven of the N ultrasound transducers 48 are shifted one by one (for each ultrasound transducer 48). Specifically, the series of processes start to be performed first on m transducers to be driven that are positioned on both sides of the ultrasound transducer 48 positioned at one end among the N ultrasound transducers 48. Then, the series of processes are repeated whenever the positions of the transducers to be driven are shifted as an open channel is switched by the multiplexer 140. Finally, the series of processes are repeatedly performed a total of N times up to m transducers to be driven that are positioned on both sides of the ultrasound transducer 48 positioned at the other end among the N ultrasound transducers 48.

The backing material layer 54 supports the respective ultrasound transducers 48 of the ultrasound transducer array 50 from the back side. Further, the backing material layer 54 has a function to attenuate ultrasound, which propagates toward the backing material layer 54, of ultrasound generated from the ultrasound transducer 48 or ultrasound (echoes) reflected by the portion to be observed. A backing material is formed of a material having stiffness, such as hard rubber, and ultrasound attenuation materials (ferrite, ceramics, and the like) are added to the backing material as necessary.

The acoustic matching layer 74 is superimposed on the ultrasound transducer array 50, and is provided for the matching of acoustic impedance between a patient's body and the ultrasound transducers 48. Since the acoustic matching layer 74 is provided, the transmittance of ultrasound can be increased. Various organic materials of which the values of acoustic impedance are closer to the value of acoustic impedance of a patient's body than the value of acoustic impedance of the piezoelectric element of the ultrasound transducer 48 can be used as the material of the acoustic matching layer 74. Specifically, examples of the material of the acoustic matching layer 74 include an epoxy-based resin, silicone rubber, polyimide, polyethylene, and the like.

The acoustic lens 76 superimposed on the acoustic matching layer 74 is to focus ultrasound, which is generated from the ultrasound transducer array 50, on a portion to be observed. The acoustic lens 76 is made of, for example, a silicone-based resin (millable silicone rubber (HTV rubber), liquid silicone rubber (RTV rubber), and the like), a butadiene-based resin, a polyurethane-based resin, and the like; and the powder of a titanium oxide, alumina, silica, or the like is mixed as necessary.

The FPC 60 is electrically connected to the electrodes of the respective ultrasound transducers 48. One end of each of the plurality of coaxial cables 56 is wired to the FPC 60. Further, in a case where the ultrasound endoscope 12 is connected to the ultrasound observation device 14 through the connector 32a for ultrasound, the other end (the end opposite to the FPC 60) of each of the plurality of coaxial cables 56 is electrically connected to the ultrasound observation device 14.

Endoscope Observation Part 38

The endoscope observation part 38 is a part that is provided to acquire endoscopic images, and is disposed in the distal end part 40 of the insertion unit 22 to be closer to the proximal end side than the ultrasound observation part 36. As shown in FIGS. 2 and 3, the endoscope observation part 38 includes an observation window 82, an objective lens 84, a solid image pickup element 86, illumination windows 88, a washing nozzle 90, a wiring cable 92, and the like.

The observation window 82 is mounted on the distal end part 40 of the insertion unit 22 so as to be inclined with respect to the axial direction (the longitudinal direction of the insertion unit 22). Light, which is reflected by an observation object-adjacent portion and is incident from the observation window 82, is made to form an image on the image pickup surface of the solid image pickup element 86 by the objective lens 84.

The solid image pickup element 86 photoelectrically converts light that is reflected by the observation object-adjacent portion, passes through the observation window 82 and the objective lens 84, and forms an image on the image pickup surface; and outputs image pickup signals. A charge coupled device (CCD), a complementary metaloxide semiconductor (CMOS), and the like can be used as the solid image pickup element 86. The image pickup signals, which are output from the solid image pickup element 86, are transmitted to the endoscope processor 16 through the wiring cable 92, which extends up to the operation unit 24 from the insertion unit 22, by the universal cord 26.

The illumination windows 88 are provided at positions on both sides of the observation window 82. The emission ends of the light guides (not shown) are connected to the illumination windows 88. The light guides extend up to the operation unit 24 from the insertion unit 22, and the incident ends of the light guides are connected to the light source device 18 connected through the universal cord 26. Illumination light generated by the light source device 18 is transmitted to the light guides, and is applied to the observation object-adjacent portion from the illumination windows 88.

The washing nozzle 90 is an ejection hole that is formed at the distal end part 40 of the insertion unit 22 to wash the surfaces of the observation window 82 and the illumination windows 88, and air or washing liquid is ejected to the observation window 82 and the illumination windows 88 from the washing nozzle 90. In this embodiment, washing liquid ejected from the washing nozzle 90 is water, particularly, degassed water. However, washing liquid is not particularly limited, and may be other liquid, for example, usual water (water not degassed).

Water Supply Tank 21a and Suction Pump 21b

The water supply tank 21a is a tank storing degassed water, and is connected to the connector 32c for a light source through an air/water supply tube 34a. Degassed water is used as washing liquid that is to be ejected from the washing nozzle 90.

The suction pump 21b sucks aspirate (including degassed water supplied for washing), which is present in the body cavity, through the treatment tool outlet 44. The suction pump 21b is connected to the connector 32c for a light source through a suction tube 34b. The ultrasound endoscope system 10 may comprise an air supply pump that supplies air to a predetermined destination to which air is to be supplied, and the like.

The treatment tool channel 45 and the air/water supply pipe lines (not shown) are provided in the insertion unit 22 and the operation unit 24.

The treatment tool channel 45 allows a treatment tool insertion opening 30, which is provided at the operation unit 24, to communicate with the treatment tool outlet 44. Further, the treatment tool channel 45 is connected to a suction button 28b provided on the operation unit 24. The suction button 28b is connected to the suction pump 21b in addition to the treatment tool channel 45.

One end of each air/water supply pipe line communicates with the washing nozzle 90, and the other end thereof is connected to an air/water supply button 28a provided on the operation unit 24. The air/water supply button 28a is connected to the water supply tank 21a in addition to the air/water supply pipe lines.

Operation Unit 24

The operation unit 24 is a unit that is operated by an operator at the time of start of ultrasound diagnosis, during diagnosis, at the time of end of diagnosis, and the like; and one end of the universal cord 26 is connected to one end of the operation unit 24. Further, as shown in FIG. 1, the operation unit 24 includes an air/water supply button 28a, a suction button 28b, a pair of angle knobs 29, and a treatment tool insertion opening (forceps port) 30.

In a case where each of the pair of angle knobs 29 is rotationally moved, the bendable part 42 is remotely operated to be bent and deformed. The distal end part 40 of the insertion unit 22, which is provided with the ultrasound observation part 36 and the endoscope observation part 38, can be made to face in a desired direction by the deformation operation of the bendable part 42.

The treatment tool insertion opening 30 is a hole that is formed to allow a treatment tool (not shown), such as forceps, to be inserted thereinto, and communicates with the treatment tool outlet 44 through the treatment tool channel 45. The treatment tool inserted into the treatment tool insertion opening 30 passes through the treatment tool channel 45 and is then introduced into the body cavity from the treatment tool outlet 44.

The air/water supply button 28a and the suction button 28b are two-stage switching push buttons, and are operated to switch the opening and closing of a pipe line provided in each of the insertion unit 22 and the operation unit 24.

Configuration of Ultrasound Observation Device 14

The ultrasound observation device 14 causes the ultrasound transducer unit 46 to transmit and receive ultrasound and converts reception signals, which are output from the ultrasound transducers 48 (in detail, elements to be driven) at the time of reception of ultrasound, into images to generate ultrasound images. Further, the ultrasound observation device 14 displays the generated ultrasound images on the monitor 20.

As shown in FIG. 4, the ultrasound observation device 14 includes a multiplexer 140, a reception circuit 142, a transmission circuit 144, an A/D converter 146, an application specific integrated circuit (ASIC) 148, a cine memory 150, a central processing unit (CPU) 152, a digital scan converter (DSC) 154, a timer control unit 168, a recording timing-management unit 170, a recording pattern-generation unit 172, an image analysis unit 174, an automatic storage control unit 176, an image recording unit 178, and an image playback unit 180.

The reception circuit 142 and the transmission circuit 144 are electrically connected to the ultrasound transducer array 50 of the ultrasound endoscope 12. The multiplexer 140 selects up to m transducers to be driven from N ultrasound transducers 48, and opens the channels of the transducers to be driven.

The transmission circuit 144 includes a field programmable gate array (FPGA), a pulser (pulse generation circuit 158), a switch (SW), and the like, and is connected to the MUX (multiplexer 140). An application specific integrated circuit (ASIC) may be used instead of the FPGA.

The transmission circuit 144 is a circuit that supplies drive voltages for the transmission of ultrasound to the transducers to be driven selected by the multiplexer 140 according to control signals sent from the CPU 152 to transmit ultrasound from the ultrasound transducer unit 46. The drive voltages are pulsed voltage signals (transmission signals), and are applied to the electrodes of the transducers to be driven through the universal cord 26 and the coaxial cables 56.

Since the transmission circuit 144 includes the pulse generation circuit 158 that generates transmission signals on the basis of the control signals, the transmission circuit 144 drives the plurality of ultrasound transducers 48 using the pulse generation circuit 158 by the control of the CPU 152 to generate transmission signals for generating ultrasound and supplies the transmission signals to the plurality of ultrasound transducers 48. In more detail, in a case where an ultrasound diagnosis is to be made, the transmission circuit 144 generates transmission signals having drive voltages for ultrasound diagnosis using the pulse generation circuit 158 by the control of the CPU 152.

The reception circuit 142 is a circuit receiving electrical signals, that is, reception signals output from the transducers to be driven which have received ultrasound (echoes). Further, the reception circuit 142 amplifies the reception signals, which are received from the ultrasound transducer 48, according to the control signals sent from the CPU 152 and delivers the amplified signals to the A/D converter 146. The A/D converter 146 is connected to the reception circuit 142, converts the reception signals received from the reception circuit 142 into digital signals from analog signals, and outputs the converted digital signals to the ASIC 148.

The ASIC 148 is connected to the A/D converter 146; and includes a phase matching section 160, a B mode image-generation section 162, a PW mode image-generation section 164, a CF mode image-generation section 166, and a memory controller 151 as shown in FIG. 4.

The above-mentioned functions (specifically, the phase matching section 160, the B mode image-generation section 162, the PW mode image-generation section 164, the CF mode image-generation section 166, and the memory controller 151) are realized by a hardware circuit, such as the ASIC 148, in this embodiment, but the invention is not limited thereto. The above-mentioned functions may be realized by the cooperation of a central processing unit (CPU) and software (computer program) causing various kinds of data processing to be performed.

The phase matching section 160 performs processing for giving delay time to the reception signals (received data), which are converted into digital signals by the A/D converter 146, to phase and add the received data (adding the received data after matching phases). Sound ray signals in which the focus of ultrasound echoes has been narrowed are generated by phasing-addition processing.

The B mode image-generation section 162, the PW mode image-generation section 164, and the CF mode image-generation section 166 generate ultrasound images on the basis of electrical signals that are output from the transducers to be driven among the plurality of ultrasound transducers 48 in a case where the ultrasound transducer unit 46 receives ultrasound (strictly speaking, sound ray signals generated in a case where received data are phased and added).

The B mode image-generation section 162 is an image-generation section that generates a B mode image which is the tomographic image of the inside (the inside the body cavity) of a patient. The B mode image-generation section 162 corrects the attenuation, which is caused by a propagation distance, of sound ray signals, which are sequentially generated, by sensitivity time gain control (STC) according to the depth of a position where ultrasound is reflected. Further, the B mode image-generation section 162 performs envelope detection processing and Log (logarithmic) compression processing on the corrected sound ray signals to generates a B mode image (image signals).

The PW mode image-generation section 164 is an image-generation section that generates an image displaying a blood flow rate in a predetermined direction. The PW mode image-generation section 164 performs fast Fourier transform on a plurality of sound ray signals, which correspond to the same direction, among the sound ray signals, which are sequentially generated by the phase matching section 160, to extract frequency components. After that, the PW mode image-generation section 164 calculates a blood flow rate from the extracted frequency components, and generates a PW mode image (image signals) displaying the calculated blood flow rate.

The CF mode image-generation section 166 is an image-generation section that generates an image displaying information about a blood flow in a predetermined direction. The CF mode image-generation section 166 obtains the autocorrelation of a plurality of sound ray signals, which correspond to the same direction, among the sound ray signals, which are sequentially generated by the phase matching section 160, to generate image signals that show information about a blood flow. After that, the CF mode image-generation section 166 generates a CF mode image (image signals) as a color image where the information about a blood flow is superimposed on the B mode image generated by the B mode image-generation section 162, on the basis of the image signals.

The memory controller 151 stores the image signals, which are generated by the B mode image-generation section 162, the PW mode image-generation section 164, or the CF mode image-generation section 166, in the cine memory 150.

The DSC 154 is connected to the ASIC 148; converts the image signals, which are generated by the B mode image-generation section 162, the PW mode image-generation section 164, or the CF mode image-generation section 166, into image signals following a scanning method for usual television signals (raster conversion); and performs various kinds of necessary image processing, such as gradation processing, on the image signals and then outputs the image signals to the monitor 20.

The cine memory 150 has a capacity that is required to store image signals corresponding to one frame or several frames. Image signals generated by the ASIC 148 are output to the DSC 154, and are also stored in the cine memory 150 by the memory controller 151. In the freeze mode, the memory controller 151 reads out the image signals stored in the cine memory 150 and outputs the image signals to the DSC 154. Accordingly, ultrasound images (static images) based on the image signals read out from the cine memory 150 are displayed on the monitor 20.

The CPU 152 functions as a control unit that controls the respective parts of the ultrasound observation device 14; is connected to the reception circuit 142, the transmission circuit 144, the A/D converter 146, the ASIC 148, the timer control unit 168, the recording timing-management unit 170, the recording pattern-generation unit 172, the automatic storage control unit 176, the image playback unit 180, and the like; and controls these components. Specifically, the CPU 152 is connected to the operation console 100, and controls the respective parts of the ultrasound observation device 14 according to examination information, control parameters, and the like input through the operation console 100.

Further, in a case where the ultrasound endoscope 12 is connected to the ultrasound observation device 14 through the connector 32a for ultrasound, the CPU 152 automatically recognizes the ultrasound endoscope 12 by a method, such as Plug and Play, such as PnP.

Here, the multiplexer 140, the reception circuit 142, the transmission circuit 144, the A/D converter 146, the ASIC 148, the cine memory 150, the CPU 152, and the DSC 154 form an ultrasound image-generation unit that drives the plurality of ultrasound transducers 48 of the ultrasound transducer unit 46 provided in the distal end part 40 of the insertion unit 22 of the ultrasound endoscope 12 to cause the ultrasound transducers 48 to transmit and receive ultrasound and generates ultrasound images from the reception signals of the ultrasound.

The timer control unit 168 includes a timer 182 and controls the measurement of a time, which is performed by the timer 182, by the control of the CPU 152 according to an instruction given from a user.

Specifically, the timer control unit 168 starts or stops the measurement of time, which is performed by the timer 182, by the control of the CPU 152 according to an instruction given from a user. Further, after ultrasound images, which are picked up at points of time when all measurement times included in a recording pattern to be described later have elapsed, are recorded in the image recording unit 178, the timer control unit 168 stops the measurement of time that is performed by the timer 182.

The recording timing-management unit 170 holds a plurality of kinds of recording patterns each of which includes a plurality of measurement times starting to be measured from a trigger timing, and selects one recording pattern from the plurality of kinds of recording patterns by the control of the CPU 152 according to an instruction given from a user.

Further, the recording timing-management unit 170 holds a new recording pattern created according to an instruction given from a user, or changes at least one of the plurality of measurement times included in one recording pattern according to an instruction given from a user.

The trigger timing is a start timing to start measuring a plurality of measurement times included in one recording pattern.

Furthermore, at least one recording pattern of the plurality of kinds of recording patterns held by the recording timing-management unit 170 may include a determination flag that causes the ultrasound endoscope system 10 to determine a recording timing to record the ultrasound image in the image recording unit 178.

Recording patterns, which are different from each other in terms of the values of measurement times, the number of measurement times, whether or not a determination flag is present, and the like, can be used as the recording patterns according to gender, age, weight, disease, a portion to be observed, and the like.

The recording pattern-generation unit 172 generates a recording pattern by the control of the CPU 152 on the basis of at least one of information about a patient, information about a patient's portion to be observed, or information about the setting of the ultrasound endoscope system 10 that is input according to an instruction given from a user.

The information about a patient includes the gender, the height, the weight, the age, the disease, and the like of a patient. The information about a patient's portion to be observed includes the pancreas, the gallbladder, the liver, the kidney, and the like. The information about the setting of the ultrasound diagnostic system includes the type of a probe, the frequency of an ultrasound beam, the conditions of the signal processing of the reception signals, and the like. Information that is used by the recording pattern-generation unit 172 to generate a recording pattern is not particularly limited, and various kinds of information can be used as the information.

The recording pattern-generation unit 172 learns a relationship between a recording pattern and at least one of the information about a patient, the information about a patient's portion to be observed, or the information about the setting of the ultrasound endoscope system 10 in advance with regard to a plurality of recording patterns; and generates an optimum recording pattern corresponding to at least one of the information about a patient, the information about a patient's portion to be observed, or the information about the setting of the ultrasound endoscope system 10, which is input according to an instruction given from a user, on the basis of the results of the learning.

A learning method is not particularly limited. For example, deep learning, which uses a hierarchical neural network and is an example of machine learning as one of techniques of artificial intelligence (AI), and the like can be used as the learning method. Machine learning other than deep learning may be used, the technique of artificial intelligence other than machine learning may be used, and a learning method other than the technique of artificial intelligence may be used.

The image analysis unit 174 includes a temporary storage area 184. In a case where a determination flag is included in one recording pattern selected by the recording timing-management unit 170, the image analysis unit 174 stores ultrasound images, which are generated by the ultrasound image-generation unit, in the temporary storage area 184 from the trigger timing. Further, the image analysis unit 174 analyzes the ultrasound images stored in the temporary storage area 184, and determines a recording timing to record the above-mentioned ultrasound image in the image recording unit 178 on the basis of the results of the analysis.

The automatic storage control unit 176 causes the image recording unit 178 to record an ultrasound image, which is picked up at a point of time when each of the plurality of measurement times has elapsed, among the plurality of ultrasound images, which are continuously generated by the ultrasound image-generation unit, by the control of the CPU 152 whenever each of the plurality of measurement times included in one recording pattern selected by the recording timing-management unit 170 elapses from the trigger timing.

The automatic storage control unit 176 can use any trigger timing, and can use, as the trigger timing, for example, a timing to give a contrast medium to a patient, a timing to start the measurement of a time performed by the timer 182, or the like.

The image recording unit 178 records at least one ultrasound image among the plurality of ultrasound images, which are continuously generated by the ultrasound image-generation unit, by the control of the automatic storage control unit 176.

The image recording unit 178 is, for example, a storage device, such as a semiconductor memory.

The image playback unit 180 causes the monitor 20 to simultaneously display the plurality of ultrasound images, which are recorded in the image recording unit 178, side by side by the control of the CPU 152 according to an instruction given from a user.

Further, the image playback unit 180 causes the monitor 20 to display a thumbnail image of an ultrasound image recorded in the image recording unit 178 by the control of the CPU 152 according to an instruction given from a user whenever the ultrasound image is recorded in the image recording unit 178, or causes the monitor 20 to display a graph showing a relationship between a time elapsed from the trigger timing and an average brightness value of a region of interest (ROI) of the ultrasound image.

Example of Operation of Ultrasound Endoscope System 10

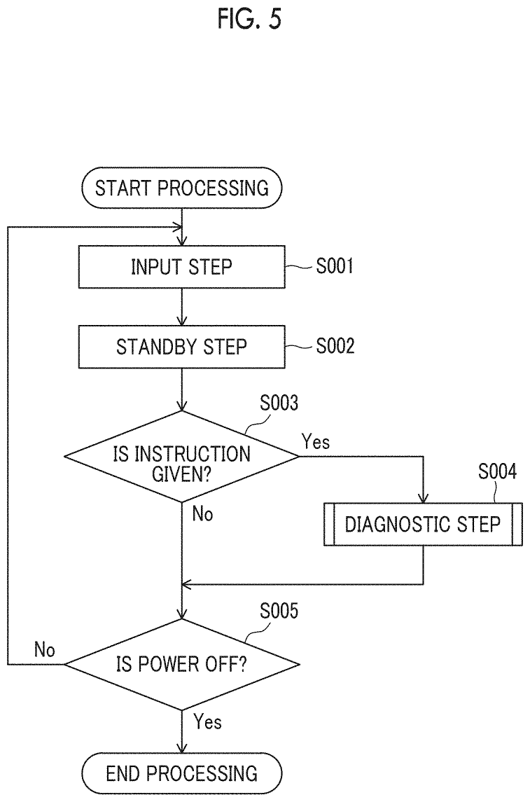

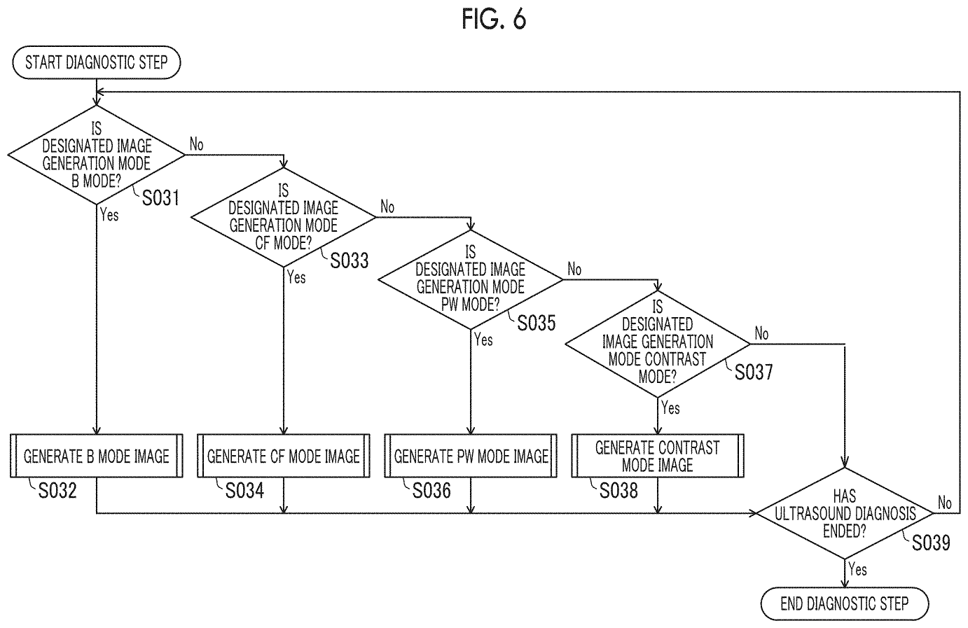

Next, the flow of a series of processing (hereinafter, referred to as diagnostic processing) related to ultrasound diagnosis will be described as an example of the operation of the ultrasound endoscope system 10 with reference to FIGS. 5 and 6. FIG. 5 is a flowchart showing the flow of diagnostic processing using the ultrasound endoscope system 10. FIG. 6 is a flowchart showing the procedure of a diagnostic step of the diagnostic processing.

In a case where power is applied to the respective parts of the ultrasound endoscope system 10 in a state where the ultrasound endoscope 12 is connected to the ultrasound observation device 14, the endoscope processor 16, and the light source device 18, diagnostic processing is started with the application of power as a trigger. In the diagnostic processing, an input step is performed (S001) first as shown in FIG. 5. In the input step, an operator inputs examination information, control parameters, and the like through the operation console 100. In a case where the input step is completed, a standby step is performed (S002) until an instruction to start diagnosis is given.

Then, in a case where an instruction to start diagnosis is given from the operator (Yes in S003), the CPU 152 controls the respective parts of the ultrasound observation device 14 to perform a diagnostic step (S004). The diagnostic step proceeds along a flow shown in FIG. 6. In a case where a designated image generation mode is a B mode (Yes in S031), the CPU 152 controls the respective parts of the ultrasound observation device 14 so that a B mode image is generated (S032). Further, in a case where the designated image generation mode is not the B mode (No in S031) and is a CF mode (Yes in S033), the CPU 152 controls the respective parts of the ultrasound observation device 14 so that a CF mode image is generated (S034). In a case where the designated image generation mode is not the CF mode (No in S033) and is a PW mode (Yes in S035), the CPU 152 controls the respective parts of the ultrasound observation device 14 so that a PW mode image is generated (S036). In a case where the designated image generation mode is not the PW mode (No in S035) and is a contrast mode (Yes in S037), the CPU 152 controls the respective parts of the ultrasound observation device 14 so that a contrast mode image is generated (S038). In a case where the designated image generation mode is not the contrast mode (No in S037), the diagnostic processing proceeds to Step S039.

Then, the CPU 152 determines whether or not ultrasound diagnosis has ended (S039). In a case where the ultrasound diagnosis has not ended (No in S039), the diagnostic processing returns to the diagnostic step S031 and the generation of an ultrasound image in each image generation mode is repeatedly performed until a condition for ending diagnosis is satisfied. Examples of the condition for ending diagnosis include an instruction to end diagnosis that is given from the operator through the operation console 100.

On the other hand, in a case where the condition for ending diagnosis is satisfied and the ultrasound diagnosis has ended (Yes in S039), the diagnostic step ends.

Then, returning to FIG. 5, in a case where the power of the respective parts of the ultrasound endoscope system 10 is off (Yes in S005), the diagnostic processing ends. On the other hand, in a case where a state where the power of the respective parts of the ultrasound endoscope system 10 is on is maintained (No in S005), the diagnostic processing returns to the input step S001 and the above-mentioned respective steps of the diagnostic processing are repeated.

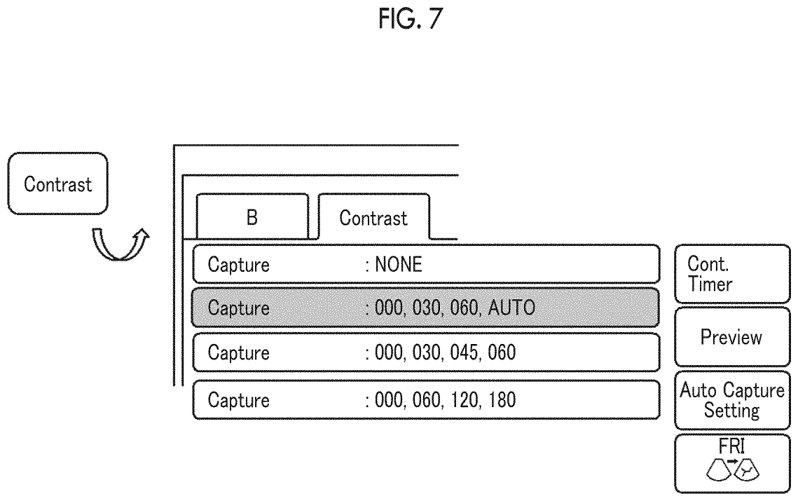

Next, a setting screen for the contrast mode will be described with reference to FIG. 7.

FIG. 7 is a conceptual diagram of an embodiment showing the screen of an operation panel of the operation console. The operation panel shown in FIG. 7 is a touch panel, and a user can input an instruction given from the user to operate the ultrasound endoscope system 10 by pressing various buttons displayed on the operation panel.

Before the setting screen for the contrast mode is displayed, a contrast mode button (Contrast) used to designate the contrast mode is displayed on the operation panel. Although not shown, a B mode button used to designate the B mode, a CF mode button used to designate the CF mode, a PW mode button used to designate the PW mode, and the like are displayed on the operation panel in addition to the contrast mode button. Accordingly, the user can designate a desired ultrasound image generation mode by pressing one button among these buttons.

In a case where the user presses the contrast mode button (Contrast) displayed on the left side in FIG. 7, an ultrasound image generation mode is set to the contrast mode. In a case where an ultrasound image generation mode is set to the contrast mode, a setting screen for the B mode and a setting screen for the contrast mode are displayed on the operation panel in the form of a tab as shown on the right side in FIG. 7. In a case where the user presses a tab (B) of the setting screen for the B mode, the setting screen for the B mode is displayed. In a case where the user presses a tab (Contrast) of the setting screen for the contrast mode, the setting screen for the contrast mode is displayed.

A plurality of kinds of recording pattern buttons (Capture . . . ) are displayed side by side in a vertical direction over the left portion of the setting screen for the contrast mode from the central portion thereof. Each of the recording pattern buttons includes a plurality of measurement times that start to be measured from the trigger timing, and may further include a determination flag.

For example, “NONE” is displayed on the first recording pattern button. “NONE” means that this recording pattern is the recording pattern of which a measurement time is not yet set. “000, 030, 060, AUTO” are displayed on the recording pattern button that is the second from the top. “000, 030, 060” means that times measured from the trigger timing are 0 sec, 30 sec, and 60 sec, respectively. Further, 0 sec is a moment of the trigger timing, and means that an ultrasound image corresponding to a state where a contrast medium does not reach a portion to be observed is acquired. “AUTO” is a determination flag. The same applies to the recording pattern buttons that are the third and the fourth from the top.

A timer start/stop button (Cont. Timer), a preview button (Preview), a measurement time-setting button (Auto Capture Setting), and a contrast medium-removal button (FRI) are displayed at the right portion of the operation panel in this order from the top.

The timer start/stop button is a toggle button that is used to start/stop the measurement of a time performed by the timer 182. In a case where the user presses the timer start/stop button and the measurement of a time performed by the timer 182 is started, the measurement of a time performed by the timer 182 is not stopped even though an ultrasound image generation mode is changed to the other ultrasound image generation mode from the contrast mode until the user presses the timer start/stop button one more and the measurement of a time performed by the timer 182 is stopped.

The preview button is a button that is used to cause the monitor 20 to simultaneously display the plurality of ultrasound images recorded in the image recording unit 178 in the contrast mode side by side.

The measurement time-setting button is a button that is used to create a new recording pattern according to an instruction given from the user or to change at least one of the plurality of measurement times included in one recording pattern selected by the recording timing-management unit 170. That is, the user can manually create a new recording pattern and hold the new recording pattern in the recording timing-management unit 170, or can manually change the measurement time, which is included in the existing recording pattern, to a desired value. In addition, the user can cause the recording pattern-generation unit 172 to generate an optimum recording pattern and can set the optimum recording pattern.

The contrast medium-removal button is a button that is used to remove the contrast medium given to a patient. A contrast medium is formed of bubbles. Accordingly, in a case where ultrasound having high sound pressure is transmitted to the contrast medium given to a patient, the bubbles of the contrast medium can be broken and removed. Therefore, it is possible to observe an aspect where a contrast medium flows in again on the screen.

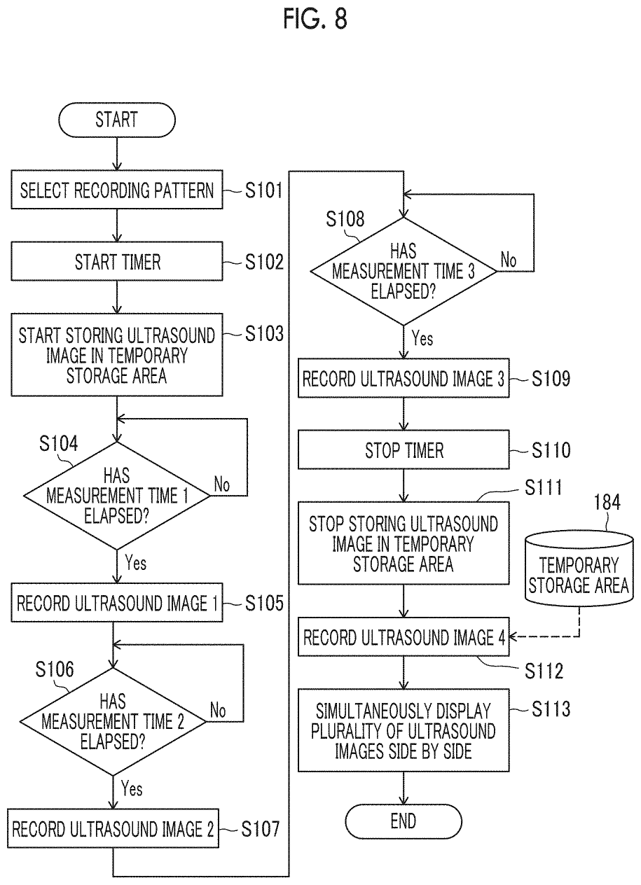

Next, the operation of the ultrasound endoscope system 10 in a case where the ultrasound image is to be observed in the contrast mode will be described with reference to a flowchart of FIG. 8.

The user inserts the insertion unit 22 of the ultrasound endoscope 12 into a patient's body cavity, and drives the plurality of ultrasound transducers 48 of the ultrasound transducer unit 46 to cause the ultrasound transducers 48 to transmit and receive ultrasound to and from a portion to be observed.

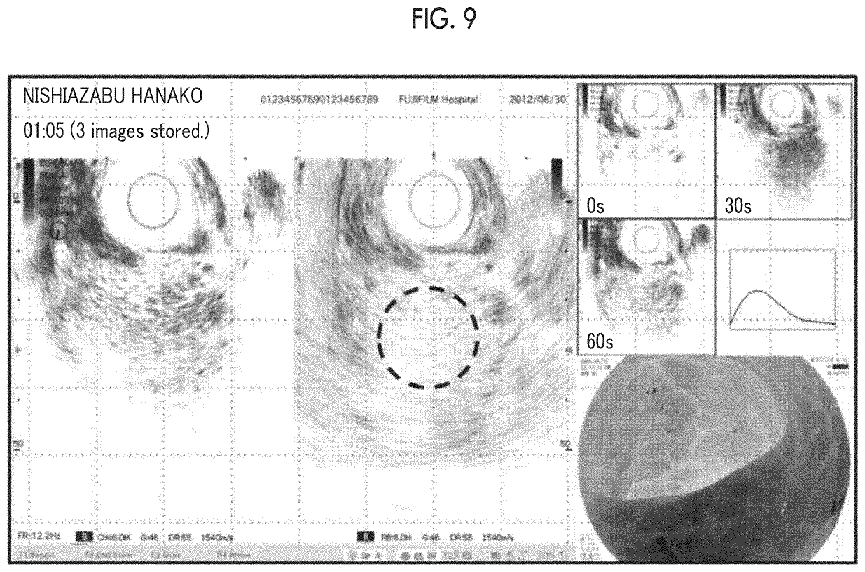

In response to this, the ultrasound images of the portion to be observed are continuously generated from the reception signals of the ultrasound by the ultrasound image-generation unit. The ultrasound images, which are continuously generated, are displayed on the monitor 20 as a video in real time in, for example, a live mode as shown in FIG. 9.

FIG. 9 is a conceptual diagram of an embodiment showing ultrasound images that are displayed on the monitor 20 in the live mode. Two ultrasound images are displayed side by side in a lateral direction over the left portion of the screen of the monitor 20 from the central portion thereof, the left image is an image where echoes reflected from a contrast medium are emphasized, and the right image is a corresponding B mode image. Further, the endoscopic image of the same portion to be observed as the ultrasound image is displayed at a lower right portion, and the thumbnails of a plurality of ultrasound images recorded in the image recording unit 178 are displayed at an upper right portion. A region, which is surrounded by a circular broken line, of the ultrasound image displayed at the central portion is a region of interest that is a set by the user. The broken line of the region of interest can also be displayed on the ultrasound image displayed at the left portion.

The initial value of the region of interest is set in the ultrasound endoscope system 10 by the automatic storage control unit 176 according to the type of the ultrasound transducer unit 46, that is, a probe used in the ultrasound diagnostic system.

For example, since ultrasound is transmitted radially (in the form of an arc) from a convex probe but ultrasound is transmitted from a radial probe over the entire circumference in a radial direction of the ultrasound endoscope 12, the convex probe and the radial probe are significantly different from each other in terms of a range observable as an ultrasound image. Accordingly, in a case where the initial value of a region of interest is set according to the type of a probe, the region of interest can be set to an appropriate region according to a range observable as an ultrasound image.

After the initial value of a region of interest is set, the user can change the region of interest to any region. However, since the user does not need to change the region of interest or the amount of change in the region of interest is small in a case where an appropriate initial value of the region of interest is set in advance, there is an advantage that the observation of an ultrasound image can be started immediately.

Then, the user presses a desired recording pattern button among the plurality of kinds of recording pattern buttons displayed on the operation panel in the setting screen for the contrast mode shown in FIG. 7 to designate a recording pattern to be used in the contrast mode.

Here, the recording pattern button, which is the second from the top in FIG. 7, is designated by the user. Accordingly, 0 sec, 30 sec, and 60 sec, are included in the recording pattern, which is designated by the user, as measurement times 1, 2, and 3, and a determination flag is further included in the designated recording pattern.

In response to this, one recording pattern, which corresponds to the recording pattern designated by the user, is selected from the plurality of kinds of recording patterns, which are held by the recording timing-management unit 170, by the recording timing-management unit 170 (S101). One recording pattern selected by the recording timing-management unit 170 is input to the automatic storage control unit 176.

Then, the user gives a contrast medium to a patient and presses the timer start/stop button displayed on the operation panel.

In response to this, the measurement of a time performed by the timer 182 is started by the control of the timer control unit 168 (S102). A time measured by the timer 182 is input to the automatic storage control unit 176.

In this embodiment, 0 sec, which is a start timing to start the measurement of a time performed by the timer 182, is set as a trigger timing by the automatic storage control unit 176. That is, a trigger timing is setting while interlocking with the start of the measurement of a time performed by the timer 182. Accordingly, since both the start of the measurement of a time performed by the timer 182 and the setting of a trigger timing can be simultaneously performed by only one operation for pressing the timer start/stop button, inconvenience of individually setting these can be eliminated.

In a case where a trigger timing is set, that is, the measurement of a time performed by the timer 182 is started in a state where a determination flag is included in one recording pattern selected by the recording timing-management unit 170, ultrasound images start to be stored in the temporary storage area 184 by the image analysis unit 174 (S103).

The image analysis unit 174 may store a plurality of ultrasound images, which are continuously generated, in the temporary storage area 184 as a video, or may store ultrasound images acquired at regular intervals, for example, at an interval of 1 sec or 10 sec in the temporary storage area 184 as static images.

Then, the automatic storage control unit 176 compares a measurement time 1 (0 sec), which is included in one recording pattern selected by the recording timing-management unit 170, with a time, which is measured by the timer 182, to determine whether or not the measurement time 1 has elapsed from the trigger timing (S104).

As a result, in a case where the measurement time 1 has not elapsed (No in S104), the processing returns to Step S104 and the automatic storage control unit 176 stands by until the measurement time 1 elapses.

On the other hand, in a case where the measurement time 1 has elapsed (Yes in S104), an ultrasound image 1 picked up at a point of time when the measurement time 1 has elapsed is recorded in the image recording unit 178 by the automatic storage control unit 176 (S105).

Then, the automatic storage control unit 176 checks whether or not a measurement time 2 (30 sec) has elapsed from the trigger timing likewise (S106), and an ultrasound image 2 picked up at a point of time when the measurement time 2 has elapsed is recorded in the image recording unit 178 (S107).

After that, the automatic storage control unit 176 checks whether or not a measurement time 3 (60 sec) has elapsed from the trigger timing likewise (S108), and an ultrasound image 3 picked up at a point of time when the measurement time 3 has elapsed is recorded in the image recording unit 178 (S109).

As shown in FIG. 9, whenever the ultrasound image is recorded in the image recording unit 178, the thumbnail image of the ultrasound image recorded in the image recording unit 178 is displayed at the upper right portion of the screen of the monitor 20, that is, at the upper side of the endoscopic image by the image playback unit 180. Accordingly, the user can check the ultrasound image, which is recorded in the image recording unit 178, while observing the ultrasound image in real time.

Further, a graph showing a relationship between a time elapsed from the trigger timing and the brightness value of a region of interest of an ultrasound image is displayed below the thumbnail image displayed at the right middle portion of the screen of the monitor 20 as shown in FIG. 9. The vertical axis of the graph represents the brightness value of a region of interest, and the horizontal axis thereof represents a time elapsed from the trigger timing. This graph is sequentially drawn as a time elapses from the trigger timing. Accordingly, the user can check an aspect where the brightness value of the region of interest is changed while observing the ultrasound image in real time.

In a case where the measurement time 3 has elapsed, the measurement of a time performed by the timer 182 is stopped by the control of the timer control unit 168 (S110).

In a case where the measurement of a time performed by the timer 182 is stopped, the image analysis unit 174 stops storing the ultrasound image in the temporary storage area 184 (S111). The user can also stop the timer 182 at any timing by pressing the timer start/stop button.

After the timer 182 is stopped and the recording of the ultrasound image in the temporary storage area 184 is stopped, the ultrasound images stored in the temporary storage area 184 are analyzed by the image analysis unit 174. A recording timing to record the ultrasound image to be recorded in the image recording unit 178, among the ultrasound images stored in the temporary storage area 184, is determined on the basis of the results of the analysis. A recording timing to store an ultrasound image of which the average brightness value of a region of interest is maximum, among the ultrasound images stored in the temporary storage area 184, in the temporary storage area 184 is determined in this embodiment.

Then, in a case where a determination flag is included in one recording pattern selected by the recording timing-management unit 170, an ultrasound image 4 picked up at the recording timing determined on the basis of the results of the analysis among the ultrasound images stored in the temporary storage area 184 is recorded in the image recording unit 178 by the automatic storage control unit 176 (S112).

In a case where the ultrasound images corresponding to all the measurement times 1, 2, and 3, and the determination flag included in one recording pattern selected by the recording timing-management unit 170 are recorded in the image recording unit 178, the user then presses the preview button displayed on the operation panel.

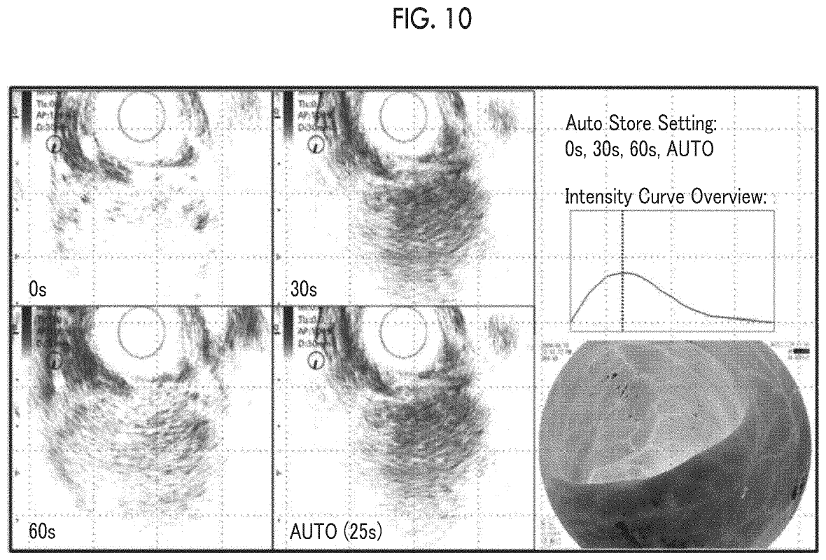

In response to this, as shown in FIG. 10, the plurality of ultrasound images recorded in the image recording unit 178 are simultaneously displayed on the monitor 20 side by side (S113). The user can identify disease by previewing the plurality of ultrasound images displayed on the monitor 20.

FIG. 10 is a conceptual diagram of an embodiment showing an aspect where a plurality of ultrasound images recorded in the image recording unit are simultaneously displayed on the monitor side by side in the contrast mode. Four ultrasound images are displayed side by side in the vertical direction and the lateral direction over the left portion of the screen of the monitor 20 from the central portion thereof. Further, information about a recording pattern is displayed at the upper right portion, and a graph showing a relationship between a time elapsed from the trigger timing and the brightness value of a region of interest of an ultrasound image is displayed at the right middle portion. Even at the time of preview, an endoscopic image can be observed at the lower right portion in real time.

As shown in FIG. 10, the ultrasound images displayed at the upper left portion, the upper right portion, and the lower left portion are ultrasound images 1, 2, and 3 that are picked up at points of time when 0 sec, 30 sec, and 60 sec have elapsed from the trigger timing as displayed as 000 s, 030 s, and 060 s. Further, the ultrasound image displayed at the lower right portion is an ultrasound image 4 of which the average brightness value of a region of interest is maximum among the ultrasound images stored in the temporary storage area 184, and a timing when the ultrasound image 4 is recorded is a point of time when 25 sec has elapsed from the trigger timing as displayed as 025 s.

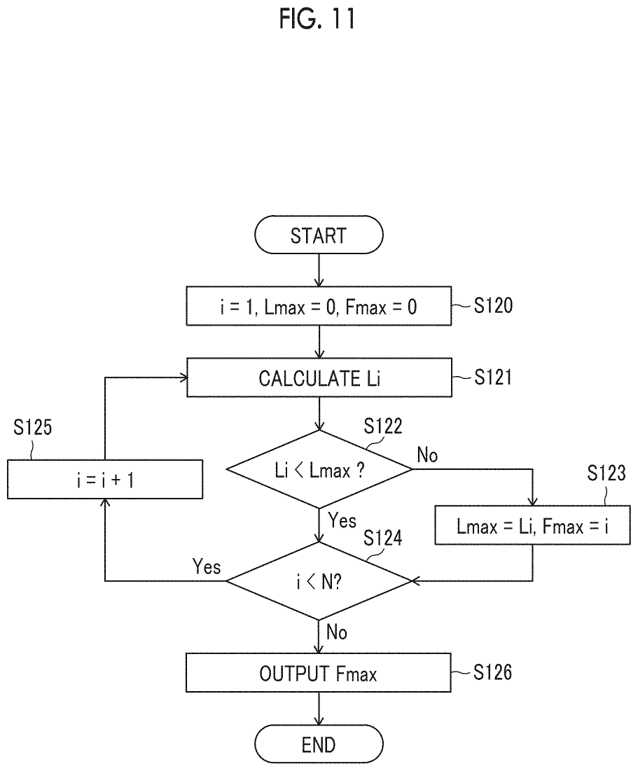

Next, the operation of the ultrasound endoscope system 10 in a case where a recording timing to record an ultrasound image in the image recording unit 178 is to be determined will be described with reference to a flowchart of FIG. 11.

In this embodiment, the frame number of an ultrasound image of which the average brightness value of a region of interest is maximum among the ultrasound images stored in the temporary storage area 184 is determined as the recording timing by the image analysis unit 174.

The total number of frames of ultrasound images obtained during the operation of the timer 182 from the start of the measurement of a time performed by the timer 182 to the stop thereof is denoted by N, a frame number in processing is denoted by i, the average brightness value of a region of interest of an ultrasound image corresponding to a frame number i is denoted by Li, the maximum value among the average brightness values of regions of interest of ultrasound images of which the total number of frames is N is denoted by Lmax, and the frame number of an ultrasound image of which the average brightness value of a region of interest is maximum is denoted by Fmax.

First, a frame number i in processing is initialized to 1 (i=1), and the maximum value Lmax among the average brightness values of regions of interest and the frame number Fmax of an ultrasound image of which the average brightness value of a region of interest is maximum are initialized to 0 (Lmax=0, Fmax=0) (S120).

Then, the average brightness value Li of a region of interest of an ultrasound image corresponding to the frame number i is calculated (S121).

After that, the average brightness value Li of the region of interest of the ultrasound image corresponding to the frame number i and the maximum value Lmax among the average brightness values of regions of interest are compared with each other (S122).

As a result, in a case where “Li<Lmax” is not satisfied, that is, the average brightness value Li of the region of interest of the ultrasound image corresponding to the frame number i is equal to or larger than the maximum value Lmax among the average brightness values of regions of interest (No in S122), Lmax is updated to Li (Lmax=Li) and Fmax is also updated to i (Fmax=i) (S123). Then, processing proceeds to Step S124.

On the other hand, in a case where “Li<Lmax” is satisfied, that is, the average brightness value Li of the region of interest of the ultrasound image corresponding to the frame number i is smaller than the maximum value Lmax among the average brightness values of regions of interest (Yes in S122), the frame number i in processing and the total number N of frames of ultrasound images are compared with each other (S124).

As a result, in a case where “i<N” is satisfied, that is, the frame number i in processing does not reach the total number N of frames of ultrasound images (Yes in S124), i is updated to i+1 (i=i+1) (S125). Then, processing returns to Step S121 and the above-mentioned operation is repeated.

On the other hand, in a case where “i<N” is not satisfied, that is, the frame number i in processing reaches the total number N of frames of ultrasound images (No in S124), the frame number Fmax of an ultrasound image of which the average brightness value of a region of interest is maximum is output as a recording timing to record an ultrasound image in the image recording unit 178 (S126).

Before the timer 182 is stopped, that is, while an ultrasound image is recorded in the temporary storage area 184, the image analysis unit 174 may analyze the ultrasound images stored in the temporary storage area 184 and determine a recording timing to record the ultrasound image to be recorded in the image recording unit 178, among the ultrasound images stored in the temporary storage area 184, on the basis of the results of the analysis.

Even in this case, in the range of the ultrasound images stored in the temporary storage area 184, likewise, the average brightness value Li of the region of interest and the maximum value Lmax among the average brightness values of regions of interest are compared with each other by the image analysis unit 174. As a result, the frame number Fmax of an ultrasound image of which the average brightness value of a region of interest is maximum is output as a recording timing to record an ultrasound image in the image recording unit 178.

A plurality of kinds of recording patterns including a plurality of elapsed times are held in the ultrasound endoscope system 10 according to gender, age, weight, disease, a portion to be observed, and the like. Accordingly, since the user can set a plurality of measurement times together by a simple operation for merely designating a desired recording pattern among the plurality of kinds of recording patterns, the user can automatically acquire an ultrasound image picked up at a point of time when each of the plurality of measurement times has elapsed from the trigger timing.

In a case where the inequality of Step S122 is appropriately changed, the image analysis unit 174 can determine at least one of, for example, a recording timing when the average brightness value of a region of interest of the ultrasound image is maximum, a recording timing when the average brightness value is minimum, a recording timing when the amount of change in an average brightness value between two ultrasound images temporally continuous is maximum, a recording timing when the variance value of the brightness value of the region of interest is maximum, a recording timing when the variance value of the brightness value is minimum, or a recording timing when the amount of change in the variance value of the brightness value between two ultrasound images temporally continuous is maximum.

Further, in a case where the recording pattern-generation unit 172 is provided as in the ultrasound endoscope system 10, the automatic storage control unit 176 may cause the image recording unit 178 to record an ultrasound image using a recording pattern, which is generated by the recording pattern-generation unit 172, instead of one recording pattern selected by the recording timing-management unit 170. Alternatively, the recording timing-management unit 170 holds a recording pattern generated by the recording pattern-generation unit 172, and a user may designate the recording pattern generated by the recording pattern-generation unit 172.

The ultrasound endoscope system 10 does not necessarily need to comprise the recording pattern-generation unit 172, and may use a recording pattern-generation device that has a function equivalent to the function of the recording pattern-generation unit 172 and is disposed outside the ultrasound endoscope system 10.

In this case, at least one of information about an examinee, information about a portion to be observed of the examinee, or information about the setting of the ultrasound diagnostic system is input to the recording pattern-generation device by the control of the CPU 152 according to an instruction given from a user.

The automatic storage control unit 176 can receive a recording pattern generated on the basis of at least one of information about an examinee, information about a portion to be observed of the examinee, or information about the setting of the ultrasound diagnostic system, which is input to the recording pattern-generation device disposed outside the ultrasound endoscope system 10, from the recording pattern-generation device; and can cause the image recording unit 178 to record the ultrasound image using the recording pattern received from the recording pattern-generation device.

Alternatively, the recording timing-management unit 170 can receive a recording pattern generated on the basis of at least one of information about an examinee, information about a portion to be observed of the examinee, or information about the setting of the ultrasound diagnostic system, which is input to the recording pattern-generation device disposed outside the ultrasound endoscope system 10, from the recording pattern-generation device; and can hold the recording pattern received from the recording pattern-generation device.

The number of measurement times included in a recording pattern is not particularly limited, and has only to be the number of measurement times required to determine whether disease is malignant or benign from the change of a brightness value over time. In this regard, it is preferable that the number of measurement times is in the range of 2 to 4.

The invention is not limited to the ultrasound endoscope system according to the embodiment, and can also be applied to various ultrasound diagnostic systems that allow the state of a portion to be observed present in the body of an examinee to be observed in a contrast mode using ultrasound.

In the system according to the embodiment of the invention, the hardware configuration of processing units, which perform various kinds of processing, such as the operation console (instruction acquisition device) 100, the timer control unit 168, the recording timing-management unit 170, the recording pattern-generation unit 172, the image analysis unit 174, the automatic storage control unit 176, and the image playback unit 180, may be dedicated hardware or may be various processors or computers executing programs.

Various processors include: a central processing unit (CPU) that is a general-purpose processor functioning as various processing units by executing software (program); a programmable logic device (PLD) that is a processor of which the circuit configuration can be changed after manufacture, such as a field programmable gate array (FPGA); a dedicated electrical circuit that is a processor having circuit configuration designed exclusively to perform specific processing, such as an application specific integrated circuit (ASIC); and the like.

One processing unit may be formed of one of these various processors, or may be formed of a combination of two or more same kind or different kinds of processors, for example, a combination of a plurality of FPGAs, a combination of an FPGA and a CPU, or the like. Further, a plurality of processing units may be formed of one processor of the various processors, or two or more of a plurality of processing units may be formed of one processor.

For example, there is an aspect where one processor is formed of a combination of one or more CPUs and software as typified by computers, such as a server and a client, and functions as a plurality of processing units. Further, there is an aspect where a processor fulfilling the functions of the entire system, which includes a plurality of processing units, by one integrated circuit (IC) chip as typified by System On Chip (SoC) or the like is used.

In addition, the hardware configuration of these various processors is more specifically electrical circuitry where circuit elements, such as semiconductor elements, are combined.

Further, the method according to the embodiment of the invention can be embodied by a program that causes a computer to perform the respective steps of the method. Furthermore, a computer-readable recording medium in which this program is recorded can also be provided.

The invention has been described in detail above, but it is natural that the invention is not limited to the above-mentioned embodiments and may have various improvements and modifications without departing from the scope of the invention.

### EXPLANATION OF REFERENCES

10: ultrasound endoscope system

12: ultrasound endoscope

14: ultrasound observation device

16: endoscope processor

18: light source device

20: monitor

21a: water supply tank

21b: suction pump

22: insertion unit

24: operation unit

26: universal cord

28a: air/water supply button

28b: suction button

29: angle knob

30: treatment tool insertion opening

32a: connector for ultrasound

32b: connector for endoscope

32c: connector for light source

34a: air/water supply tube

34b: suction tube

36: ultrasound observation part

38: endoscope observation part

40: distal end part

42: bendable part

43: soft part

44: treatment tool outlet

45: treatment tool channel

46: ultrasound transducer unit

48: ultrasound transducer

50: ultrasound transducer array

54: backing material layer

56: coaxial cable

60: FPC

74: acoustic matching layer

76: acoustic lens

82: observation window

84: objective lens

86: solid image pickup element

88: illumination window

90: washing nozzle

92: wiring cable

100: operation console

140: multiplexer

142: reception circuit

144: transmission circuit

146: A/D converter

148: ASIC

150: cine memory

151: memory controller

152: CPU

154: DSC

158: pulse generation circuit

160: phase matching section

162: B mode image-generation section

164: PW mode image-generation section

166: CF mode image-generation section

168: timer control unit

170: recording timing-management unit

172: recording pattern-generation unit

174: image analysis unit

176: automatic storage control unit

178: image recording unit

180: image playback unit

182: timer

184: temporary storage area

What is claimed is:

1. An ultrasound diagnostic system comprising:
an ultrasound image-generation unit that drives an ultrasound transducer to cause the ultrasound transducer to transmit and receive ultrasound and generates an ultrasound image from a reception signal of the ultrasound;
an instruction acquisition device that acquires an instruction input from a user;
a recording timing-management unit that holds a plurality of kinds of recording patterns each of which includes a plurality of measurement times starting to be measured from a trigger timing and selects one recording pattern from the plurality of kinds of recording patterns according to the instruction input from the user;
an image recording unit that records at least one ultrasound image among a plurality of ultrasound images continuously generated by the ultrasound image-generation unit; and
an automatic storage control unit that causes the image recording unit to record an ultrasound image, which is picked up at a point of time when each of the plurality of measurement times has elapsed, among the plurality of ultrasound images, which are continuously generated by the ultrasound image-generation unit, whenever each of the plurality of measurement times included in the one recording pattern elapses from the trigger timing.

2. The ultrasound diagnostic system according to claim 1,
wherein the automatic storage control unit receives a recording pattern generated on the basis of at least one of information about an examinee, information about a portion to be observed of the examinee, or information about setting of the ultrasound diagnostic system, which is input to a recording pattern-generation device disposed outside the ultrasound diagnostic system, from the recording pattern-generation device, and causes the image recording unit to record the ultrasound image using the recording pattern received from the recording pattern-generation device.

3. The ultrasound diagnostic system according to claim 1,
wherein the recording timing-management unit receives a recording pattern generated on the basis of at least one of information about an examinee, information about a portion to be observed of the examinee, or information about setting of the ultrasound diagnostic system, which is input to a recording pattern-generation device disposed outside the ultrasound diagnostic system, from the recording pattern-generation device, and holds the recording pattern received from the recording pattern-generation device.

4. The ultrasound diagnostic system according to claim 1, further comprising:
a recording pattern-generation unit that generates a recording pattern on the basis of at least one of information about an examinee, information about a portion to be observed of the examinee, or information about setting of the ultrasound diagnostic system input according to the instruction input from the user,
wherein the automatic storage control unit causes the image recording unit to record the ultrasound image using the recording pattern generated by the recording pattern-generation unit.

5. The ultrasound diagnostic system according to claim 1, further comprising:
a recording pattern-generation unit that generates a recording pattern on the basis of at least one of information about an examinee, information about a portion to be observed of the examinee, or information about setting of the ultrasound diagnostic system input according to the instruction input from the user,
wherein the recording timing-management unit holds the recording pattern generated by the recording pattern-generation unit.

6. The ultrasound diagnostic system according to claim 1, further comprising:
a timer control unit that includes a timer and controls measurement of a time performed by the timer,
wherein the automatic storage control unit uses, as the trigger timing, a timing to start the measurement of a time performed by the timer.

7. The ultrasound diagnostic system according to claim 1, further comprising:
an image playback unit that causes a monitor to simultaneously display a plurality of the ultrasound images, which are recorded in the image recording unit, side by side.

8. The ultrasound diagnostic system according to claim 7,
wherein the image playback unit causes the monitor to display a thumbnail image of the ultrasound image recorded in the image recording unit whenever the ultrasound image is recorded in the image recording unit.

9. The ultrasound diagnostic system according to claim 7,
wherein the image playback unit causes the monitor to display a graph showing a relationship between a time elapsed from the trigger timing and an average brightness value of a region of interest of the ultrasound image.

10. The ultrasound diagnostic system according to claim 1,
wherein at least one recording pattern of the plurality of kinds of recording patterns held by the recording timing-management unit includes a determination flag that causes the ultrasound diagnostic system to determine a recording timing to record the ultrasound image in the image recording unit.

11. The ultrasound diagnostic system according to claim 10, further comprising:
an image analysis unit that includes a temporary storage area, stores the ultrasound images in the temporary storage area from the trigger timing, analyzes the ultrasound images stored in the temporary storage area, and determines the recording timing on the basis of results of the analysis,
wherein the automatic storage control unit causes the image recording unit to record an ultrasound image, which is picked up at the recording timing determined on the basis of the results of the analysis, among the ultrasound images stored in the temporary storage area in a case where the determination flag is included in the one recording pattern.

12. The ultrasound diagnostic system according to claim 11,
wherein the image analysis unit determines at least one of a recording timing when an average brightness value of a region of interest of the ultrasound image is maximum, a recording timing when the average brightness value is minimum, a recording timing when an amount of change in the average brightness value between two ultrasound images temporally continuous is maximum, a recording timing when a variance value of a brightness value of the region of interest is maximum, a recording timing when the variance value of the brightness value is minimum, or a recording timing when an amount of change in the variance value of the brightness value between two ultrasound images temporally continuous is maximum.

13. The ultrasound diagnostic system according to claim 12,
wherein the automatic storage control unit sets an initial value of the region of interest according to a type of a probe used in the ultrasound diagnostic system.

14. The ultrasound diagnostic system according to claim 1,
wherein the recording timing-management unit further holds a new recording pattern created according to the instruction input from the user.

15. The ultrasound diagnostic system according to claim 1,
wherein the recording timing-management unit further changes at least one of the plurality of measurement times included in the one recording pattern according to the instruction input from the user.

16. A method of operating an ultrasound diagnostic system, comprising:
a step of causing an ultrasound image-generation unit to drive an ultrasound transducer to cause the ultrasound transducer to transmit and receive ultrasound and causing the ultrasound image-generation unit to generate an ultrasound image from a reception signal of the ultrasound;
a step of causing a recording timing-management unit, which holds a plurality of kinds of recording patterns each of which includes a plurality of measurement times starting to be measured from a trigger timing, to select one recording pattern from the plurality of kinds of recording patterns according to the instruction input from a user; and
a step of causing an automatic storage control unit to cause an image recording unit to record an ultrasound image, which is picked up at a point of time when each of the plurality of measurement times has elapsed, among a plurality of ultrasound images, which are continuously generated by the ultrasound image-generation unit, whenever each of the plurality of measurement times included in the one recording pattern elapses from the trigger timing.

17. The method of operating an ultrasound diagnostic system according to claim 16,
wherein at least one of information about an examinee, information about a portion to be observed of the examinee, or information about setting of the ultrasound diagnostic system is input to a recording pattern-generation device disposed in the ultrasound diagnostic system,
a recording pattern generated on the basis of at least the one of the information about the examinee, the information about the portion to be observed of the examinee, or the information about the setting of the ultrasound diagnostic system is received from the recording pattern-generation device, and
the image recording unit is caused to record the ultrasound image using the recording pattern received from the recording pattern-generation device.

18. The method of operating an ultrasound diagnostic system according to claim 16, further comprising:
a step of causing an image playback unit to cause a monitor to simultaneously display a plurality of ultrasound images, which are recorded in the image recording unit, side by side.

19. The method of operating an ultrasound diagnostic system according to claim 16,
wherein at least one recording pattern of the plurality of kinds of recording patterns held by the recording timing-management unit includes a determination flag that causes the ultrasound diagnostic system to determine a recording timing to record the ultrasound image in the image recording unit.

20. The method of operating an ultrasound diagnostic system according to claim 19, further comprising:
a step of causing an image analysis unit, which includes a temporary storage area, to store the ultrasound images in the temporary storage area from the trigger timing, to analyze the ultrasound images stored in the temporary storage area, and to determine the recording timing on the basis of results of the analysis,
wherein the image recording unit is caused to record an ultrasound image, which is picked up at the recording timing determined on the basis of the results of the analysis, among the ultrasound images stored in the temporary storage area in a case where the determination flag is included in the one recording pattern.
